(19) United States
(12) Patent Application Publication
(10) Pub. No.: US 2009/0105824 A1
(43) Pub. Date: Apr. 23, 2009

(54) SPINAL FUSION DEVICE AND ASSOCIATED METHODS

(76) Inventors: Robert J. Jones, Austin, TX (US); Kevin Dunworth, Dripping Springs, TX (US)

Correspondence Address: JOHNSON & ASSOCIATES, PO BOX 90698, AUSTIN, TX, 78709-0698, US

(21) Appl. No.: 11/875,582

(22) Filed: Oct. 19, 2007

(51) Int. Cl. A61F 2/44

(52) U.S. Cl. 623/17.16

(57) ABSTRACT

A method and apparatus is provided for use in spinal fusion procedures. An exemplary interbody fusion device includes a synthetic non-metallic radiolucent interbody spacer having an opening formed between its top and bottom surfaces. A cancellous allograft plug is configured to be disposed within the opening formed in the spacer. The cancellous allograft plug can be reconstituted with a material that will help to facilitate fusion of the vertebrae.

### FIELD OF THE INVENTION

This invention relates to the field of spinal fusion. In particular, this invention is drawn to spinal fusion devices and associated methods.

### BACKGROUND OF THE INVENTION

The spine can be considered to be a series of movable segments made up of vertebrae and discs. Due to trauma, disease, and/or aging, the spine may be subject to degeneration. This degeneration may destabilize the spine and cause pain and/or nerve damage. Medical procedures are often required to either ease back pain, repair damage, or to prevent future damage.

One procedure that is often used to treat back pain or spinal damage is spinal fusion. Spinal fusion is a surgical technique used to combine two or more adjacent vertebrae. Supplemental bone tissue is used in conjunction with the patient's natural osteoblastic processes in a spinal fusion procedure. Spinal fusion is used primarily to eliminate back pain caused by the motion of the damaged vertebrae by immobilizing adjacent vertebrae. Conditions for which spinal fusion might be done include degenerative disc disease, treatment of a spinal tumor, a vertebral fracture, scoliosis, degeneration of the disc, spondylolisthesis, or any other condition that causes instability of the spine.

There is a need for spinal fusion devices and related spinal fusion procedures that adequately treats degenerative disc disease and other spinal conditions, while providing improvements over the prior art.

### SUMMARY OF THE INVENTION

A spinal fusion device includes a spacer made from a synthetic non-metallic radiolucent material and configured to be placed between adjacent vertebrae and a cancellous allograft plug configured to be disposed within an opening formed in the spacer.

Another embodiment of the invention provides a method of forming a spinal fusion device including providing a synthetic non-metallic radiolucent cervical spacer, configuring a cancellous allograft plug to fit into an opening of the cervical spacer, wherein the plug is configured to contact edges that define the opening when the cancellous allograft plug is disposed within the opening; and wherein the synthetic non-metallic radiolucent cervical spacer and cancellous allograft plug are configured to be inserted between two adjacent vertebrae to facilitate the fusion of the two adjacent vertebrae.

One embodiment of a surgical implant includes a synthetic non-metallic radiolucent fusion bearing spacer and a cancellous allograft plug having a shape that generally conforms to the opening formed in the spacer, enabling the cancellous allograft plug to be inserted into the opening.

Another embodiment of the invention provides a method of fusing adjacent vertebrae including providing a synthetic non-metallic radiolucent interbody spacer, inserting a cancellous allograft plug into an opening of the interbody spacer, and inserting the vertebral spacer and cancellous allograft plug between two adjacent vertebrae to facilitate the fusion of the two adjacent vertebrae.

Other features and advantages of the present invention will be apparent from the accompanying drawings and from the detailed description that follows below.

### BRIEF DESCRIPTION OF THE DRAWINGS

The present invention is illustrated by way of example and not limitation in the figures of the accompanying drawings, in which like references indicate similar elements and in which:

FIG. 1 is an isometric view of one example of an interbody fusion device of the present invention.

FIG. 2 is an isometric diagram of the interbody fusion device shown in FIG. 1 installed between the end plates of two adjacent vertebrae.

FIG. 3 is an exploded view of the interbody fusion device shown in FIG. 1.

FIG. 4 is a side view of an interbody spacer.

FIG. 5 is a top view of an interbody spacer.

FIG. 6 is a bottom view of an interbody spacer.

FIG. 7 is an exploded isometric diagram illustrating an interbody fusion device before being inserted between two vertebrae.

FIG. 8 is a flowchart illustrating one example of a spinal fusion procedure.

### DETAILED DESCRIPTION

The present invention relates to spinal fusion implants and related spinal fusion procedures for use in cervical and lumbar applications. One type of spinal fusion is interbody fusion. Typically, an interbody fusion procedure places a bone graft between two adjacent vertebra in the area normally occupied by an intervertebral disc. In preparation for a spinal fusion procedure, the intervertebral disc is removed. A device may be placed between the vertebra to maintain spine alignment and disc height. Fusion then occurs between the endplates of the vertebrae. The present invention provides an interbody fusion device and related methods that provide various advantages over the prior art.

Generally, the present invention provides an interbody fusion device that may be used for cervical and lumbar interbody fusion. In one example, a first piece of the interbody fusion device is a load bearing device having an opening formed between its top and bottom surfaces. The first piece is designed to bear the axial loading from the end plates of adjacent vertebrae. A second piece of the interbody fusion device is a cancellous allograft plug configured to fit within the opening formed in the first piece. The cancellous allograft plug can be reconstituted with a material that will help to facilitate fusion of the vertebrae.

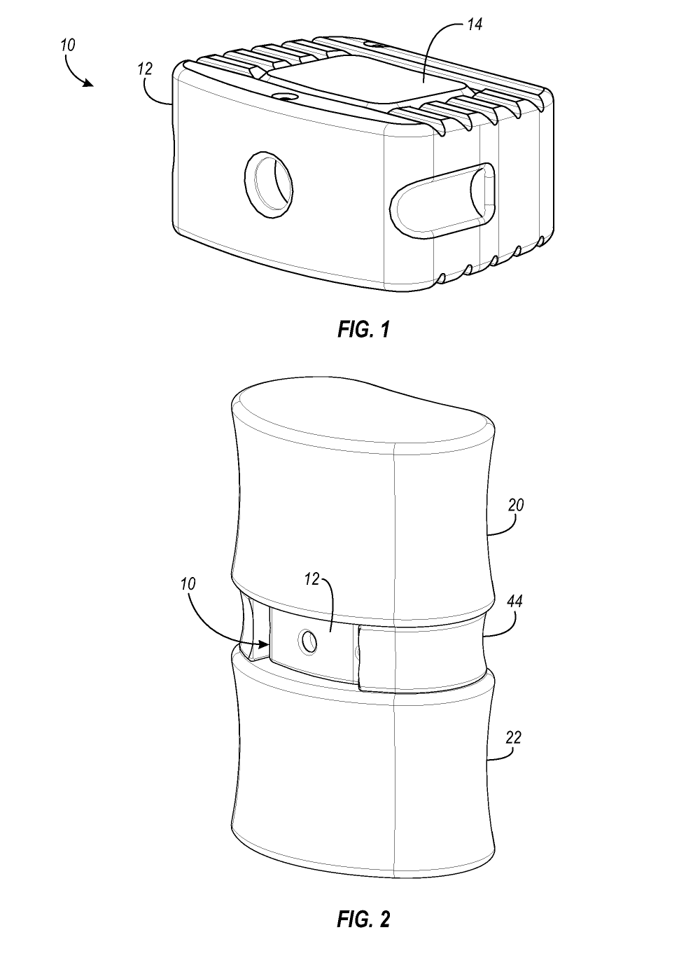

FIG. 1 is an isometric view of one example of an interbody fusion device of the present invention. FIG. 1 shows an interbody fusion device 10. The interbody fusion device 10 includes a load bearing interbody spacer 12 and a cancellous allograft plug 14, each of which are described in more detail below.

FIG. 2 is an isometric diagram of the interbody fusion device 10 shown in FIG. 1 installed between the end plates of two adjacent vertebrae 20 and 22 to facilitate the fusion of the vertebrae 20 and 22. The interbody fusion device 10 provides load bearing support as well as the proper spacing between the vertebrae 20 and 22 while fusion of the vertebrae takes place. As described in more detail below, the interbody fusion device 10 is positioned between the end plates of the vertebrae 20 and 22 within the vertebral body in the area usually occupied by the intervertebral disc.

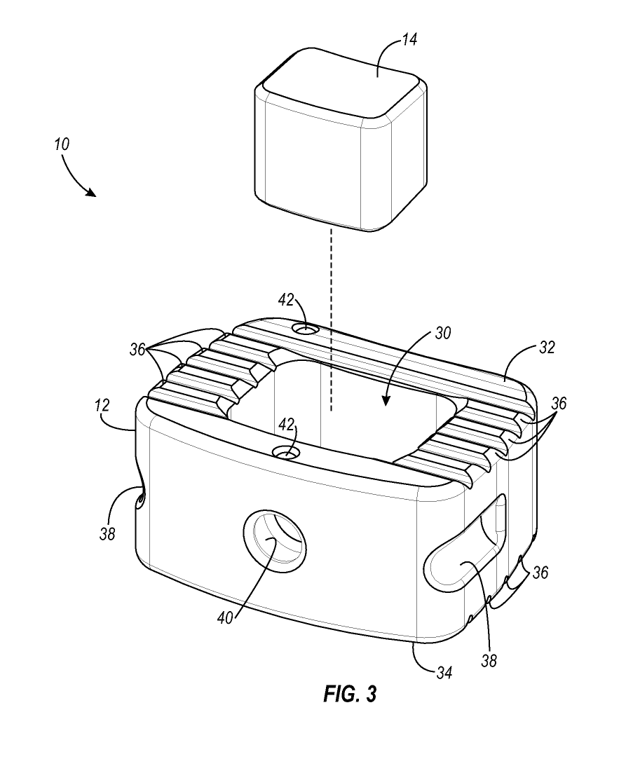

FIGS. 3-6 are views illustrating various details of one example of an interbody fusion device of the present invention. FIG. 3 is an exploded view of the interbody fusion device 10, showing the load bearing spacer 12 and the cancellous allograft plug 14 separately. The interbody fusion device 10 is shaped to fit between adjacent vertebrae in the location of the intervertebral disc. The spacer 12 has an opening 30 formed between the upper and lower surfaces 32 and 34 of the spacer 12. The opening 30 allows the allograft plug 14 to be inserted into the spacer 12, as is described in detail below.

The spacer 12 also includes a plurality of ridges 36 formed on the top and bottom surfaces 32 and 34 of the spacer 12. The ridges 36 are angled and come to a point in such a way that the ridges 36 help to hold the spacer 12 to the end plates of the vertebrae to reduce the chance of anterior migration of the implant.

If desired, one or more openings can be formed in the spacer 12 to facilitate instrumentation devices. In the example shown in FIG. 3, two lateral scallops 38 are formed on opposite sides of the load bearing device 12. A central scallop 40 is formed on the front surface of the spacer 12. The two lateral scallops 38 facilitate gripping the fusion device 10 using a bi-fed instrument grip (not shown), such as a Kerrison-style implant holder or a forceps style implant holder. The central scallop 40 facilitates manipulation of the fusion device 10 using an implant pusher (not shown). An implant pusher would typically have a dimple formed that matches the central scallop 40 to prevent slippage of the implant pusher. The lateral and central scallops 38 and 40 allow all degrees of manipulation, while not compromising superior or inferior endplate interference.

In one example, a plurality of radio opaque markers 42 (two are shown in FIG. 3, others are shown in the figures described below) are embedded into the spacer 12. In some examples, the spacer 12 is made from a radiolucent material, which allows doctors to view the nearby vertebral bodies using X-rays without the spacer 12 blocking the view of the vertebral bodies. Since the spacer 12 is made from a radiolucent material, it may be difficult for a doctor to observe the fusion device during, or after a surgery. However, the radio opaque markers 42 will show up in an X-ray. Since the positions of the markers 42 are known relative to the spacer 12, a doctor can determine the position of the fusion device 10 in an X-ray by viewing the positions of the markers 42.

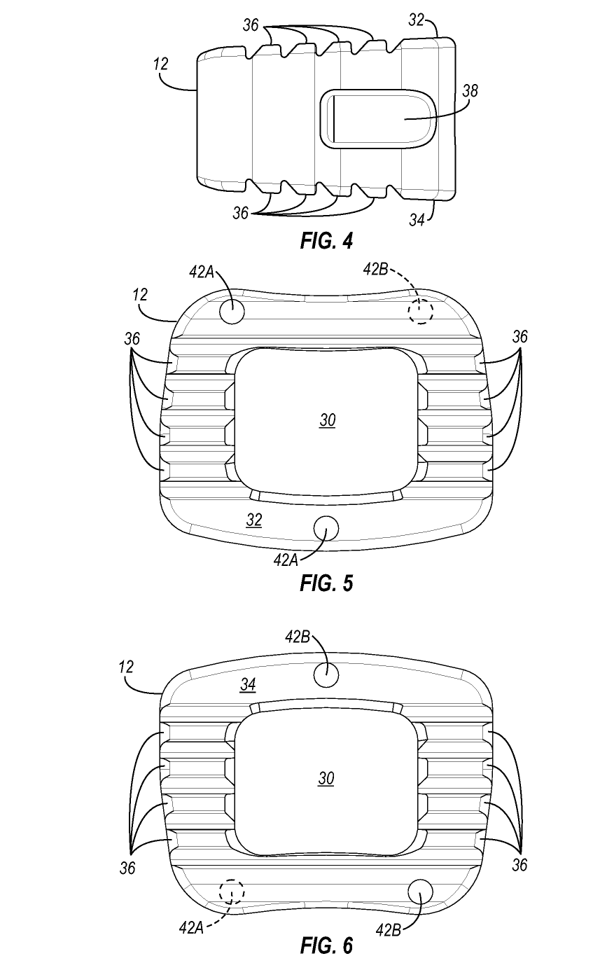

FIG. 4 is a side view of the spacer 12. FIG. 4 shows a plurality of ridges 36 formed on the upper and lower surfaces 32 and 34 of the spacer 12. FIG. 4 also shows one of the lateral scallops 38 formed on the side of the spacer. FIG. 5 shows a top view of the spacer 12. As shown, a plurality of ridges 36 are formed in the upper surface 32 of the spacer 12. FIG. 6 shows a bottom view of the spacer 12. As shown, a plurality of ridges 36 are formed in the lower surface 34 of the spacer 12.

FIGS. 5 and 6 also show an exemplary size and shape of the opening 30. The opening 30 provides a relatively large graft volume, compared to a typical device. Prior to insertion between two vertebrae, the opening 30 can be filled with the cancellous alograft plug 14. As mentioned, the plug 14 can be reconstituted using a prepared material that will help to facilitate fusion of the vertebrae. Examples of a material include bone marrow, bone morphonogenic protein (BMP), Autologous Stem Cells, etc., to facilitate fusion through the opening 30.

FIGS. 5 and 6 also show an exemplary locations for a plurality of radio opaque markers 42. In the example shown, four markers 42 are embedded into the spacer 12, with two markers being disposed at or near each of the upper and lower surfaces 32 and 34. In FIGS. 5 and 6, the markers disposed at or near the upper surface 32 correspond to reference numeral 42A and the markers disposed at or near the lower surface 34 correspond to reference numeral 42B.

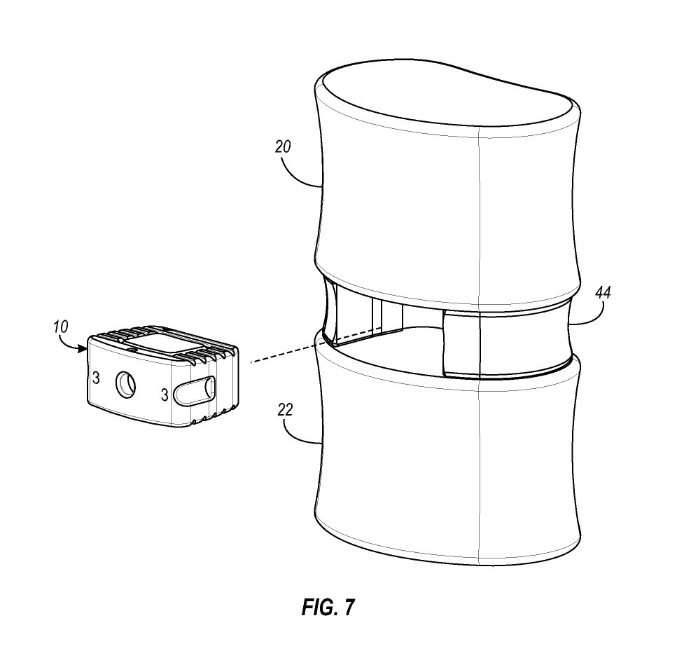

As described above, FIG. 1 is an isometric diagram of the interbody fusion device 10 with the cancellous allograft plug 14 inserted into the opening 30 formed in the spacer 12. The resulting assembly provides a load bearing structure that also allows desirable fusion of the adjacent vertebrae. Once the cancellous allograft plug 14 is reconstituted (described below) and inserted into the opening 30 of the spacer 12, the interbody fusion device can be inserted between adjacent vertebrae. Prior to the insertion of the interbody fusion device 10, the intervertebral disc is removed, so the interbody fusion device 10 can be placed between the vertebrae 20 and 22. In one example, a window is cut in the disc annulus 44. Next, portions of the nucleus pulposus are removed so that the interbody fusion device 10 can fit between the vertebrae 20 and 22 as shown in the figures. FIG. 7 is an exploded isometric diagram illustrating the interbody fusion device 10 after the plug 14 is inserted into the spacer 12 (FIG. 1), but before it is inserted between two vertebrae 20 and 22. FIG. 2 (described above) illustrates the interbody fusion device after it has been inserted between the vertebrae 20 and 22.

As mentioned, prior to insertion into the spacer 12, the cancellous allograft plug 14 is reconstituted, using a material that will help to facilitate fusion of the vertebrae. The reconstitution of the cancellous allograft plug can be accomplished using any desired technique, such as soaking the plug in the material. The reconstitution process makes the plug (or alternate carrier material) goes from a dehydrated or semi dehydrated state to a state where it is able to take on fluid and increase in mass and volume. Any desired material may be used, including bone marrow, bone morphonogenic protein (BMP), Autologous Stem Cells, etc. Note that, many materials from the body (e.g., blood, adipose tissue, muscle, organs, placenta, bone, teeth, bodily fluids, bone marrow, etc.) contain stem cells. When referring to autologous stem cells above, it is intended that autologous stem cells refers to stem cells that have been concentrated from the body.

One advantage of the present invention relates to the simplification of a spinal fusion surgery. Since the cancellous allograft plugs are pre-formed to fit into the openings of spacers (having known dimensions), the spacers and allograft plugs can be packaged or organized together prior to surgery. In other words, once a surgeon has selected a desired spacer, he or she will not have to make an effort and take time to select a plug, or to prepare custom fusion material. In one example, a spacer and a matching allograft plug are packaged together, making the surgical procedure easier. If desired, a single package can include one spacer and one plug, or a plurality of spacers and plurality of plugs. In another example, the spacers and plugs are packaged separately, with the spacers and plugs being appropriately labeled to allow a user to easily match the appropriate spacers and plugs.

When a surgeon prepares the vertebral body for the implant (e.g., by removing the disc and cleaning out the space between the vertebrae, etc.), the surgeon can determine what size and angle implant is desired. In one example, the spacers can be provided in several different predetermined heights. The plugs are each configured to be used with specific spacers. Therefore, a surgeon only need to determine the proper sized spacer to use, since each spacer corresponds to certain plugs. In one example, each type of spacers has an identifier (e.g., numbers and/or letters) on it. The identifier will tell a surgeon which plug to use with that particular spacer. FIG. 7 shows an example of how an identifier can be used. As shown, the spacer 12 in FIG. 7 has an identifier of “3.” Presumably, a surgeon selected a “3” spacer based on a desired implant height, angle, etc. Once a “3” spacer is selected, a corresponding proper plug will be known, without the surgeon having to make a separate determination.

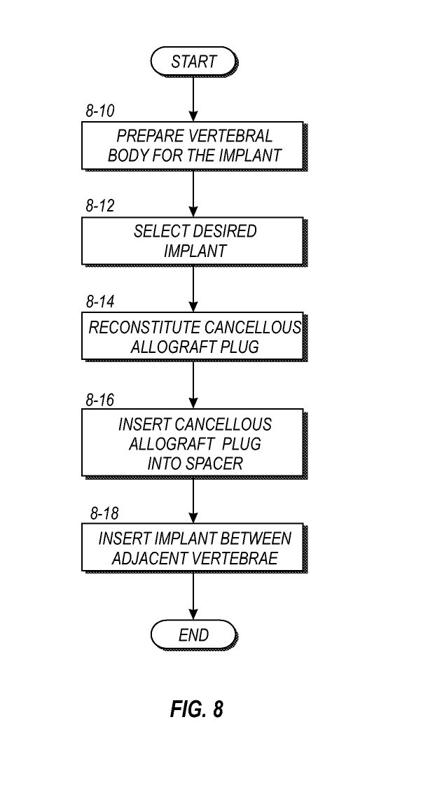

FIG. 8 is a flowchart illustrating an example of how an interbody fusion device of the present invention may be used in a spinal fusion procedure. At step 8-10, the vertebral body is prepared for the implant. For example, a window is cut in the side of the disc annulus (e.g., FIG. 7) to allow an interbody fusion implant to be inserted. The nucleus pulposus can also be cleaned out to provide room for the implant. In addition, a surgeon may scrape each vertebral body to help the fusion process. At step 8-12 a desired implant is selected. This selection can be based on factors such as the desired height between the adjacent vertebrae, the desired lordosis, etc. At step 8-14, the cancellous allograft plug is reconstituted, using a material that will promote fusion. Once the allograft plug is ready, it can be inserted into the opening of the spacer (step 8-16) (FIG. 1). In another example, the plug can first be inserted into the spacer, and then reconstituted while it is inserted into the spacer. At step 8-18, the implant is inserted between the adjacent vertebrae using the appropriate instrumentation, as desired (FIGS. 7 and 2).

The spinal fusion device of the present invention can be made from any desired materials. In one example, the spacer is made from a synthetic non-metallic radiolucent material. A radiolucent material will allow a doctor to adequately view x-rays of bones without the spacer obstructing the view. As mentioned above, one or more radio opaque markers can be embedded into the spacer to allow a doctor to view the relative position of the spacers. Examples of synthetic non-metallic radiolucent material include, but are not limited to, thermoplastic materials such as Polyetheretherketones PEEK or Polyetherketoneketone (PEKK), carbon fiber, etc. The plug can also be made from any desired carrier material. Examples of carrier material include, but are not limited to, cancellous bone, cancellous chips, Hydroxylapatite, Helos, Tricalcium phosphate (bone ash), Collagen Sponge, etc.

In the preceding detailed description, the invention is described with reference to specific exemplary embodiments thereof. Various modifications and changes may be made thereto without departing from the broader spirit and scope of the invention as set forth in the claims. The specification and drawings are, accordingly, to be regarded in an illustrative rather than a restrictive sense.

What is claimed is:

1. A spinal fusion device comprising:
a spacer configured to be placed between adjacent vertebrae, the spacer having first and second opposing surfaces, wherein the spacer is made from a synthetic non-metallic radiolucent material;
an opening formed in the spacer between the first and second opposing surfaces; and
a cancellous allograft plug configured to be disposed within the opening formed in the spacer.

2. The spinal fusion device of claim 1, further comprising a plurality of ridges formed in the first and second opposing surfaces to prevent migration of the spacer.

3. The spinal fusion device of claim 1, wherein the synthetic non-metallic radiolucent material is comprised of a thermoplastic material.

4. The spinal fusion device of claim 1, wherein the synthetic non-metallic radiolucent material is comprised of Polyetheretherketones (PEEK).

5. The spinal fusion device of claim 1, further comprising a plurality of radio opaque markers formed in the spacer to allow a user to determine the position of the spinal fusion device relative to a spine using x-rays.

6. The spinal fusion device of claim 1, wherein the cancellous allograft plug is pre-formed to closely fit within the opening formed in the cervical spacer.

7. The spinal fusion device of claim 1, wherein the cancellous allograft plug is configured to fit within the opening formed in the cervical spacer such that the cancellous allograft plug engages surfaces that form the opening in the cervical spacer.

8. A method of forming a spinal fusion device comprising:
providing a synthetic non-metallic radiolucent cervical spacer having first and second opposing surfaces and an opening formed in the cervical spacer between the first and second opposing surfaces;
configuring a cancellous allograft plug to fit into the opening of the cervical spacer, wherein the plug is configured to contact edges that define the opening when the cancellous allograft plug is disposed within the opening; and
wherein the synthetic non-metallic radiolucent cervical spacer and cancellous allograft plug are configured to be inserted between two adjacent vertebrae to facilitate the fusion of the two adjacent vertebrae.

9. The method of claim 8, further comprising reconstituting the cancellous allograft plug in a material to enhance spinal fusion.

10. The method of claim 8, further comprising forming a plurality of ridges in the first and second opposing surfaces to prevent migration of the cervical spacer.

11. The method of claim 8, wherein the synthetic non-metallic radiolucent material is comprised of a thermoplastic material.

12. The method of claim 8, wherein the synthetic non-metallic radiolucent material is comprised of Polyetheretherketones (PEEK).

13. The method of claim 8, further comprising forming a plurality of radio opaque markers in the synthetic non-metallic radiolucent cervical spacer to allow a user to determine the position of the spinal fusion device relative to a spine using x-rays.

14. A surgical implant comprising:
a synthetic non-metallic radiolucent fusion bearing spacer having top and bottom surfaces and being configured to fit between two adjacent vertebra, the spacer having an opening formed between the top and bottom surfaces; and
a cancellous allograft plug having a shape that generally conforms to the opening formed in the spacer, enabling the cancellous allograft plug to be inserted into the opening, while substantially filling the opening.

15. The surgical implant of claim 14, further comprising a plurality of ridges formed in the top and bottom surfaces to prevent migration of the spacer.

16. The surgical implant of claim 14, wherein the synthetic non-metallic radiolucent fusion bearing spacer is comprised of a thermoplastic material.

17. The surgical implant of claim 14, wherein the synthetic non-metallic radiolucent fusion bearing spacer is comprised of Polyetheretherketones (PEEK).

18. The surgical implant of claim 14, further comprising one or more radio opaque markers embedded in the surgical implant to allow a user to determine the position of the surgical implant using x-rays.

19. A method of fusing adjacent vertebrae comprising:
providing a synthetic non-metallic radiolucent interbody spacer having first and second opposing surfaces and an opening formed in the interbody spacer between the first and second opposing surfaces;
inserting a cancellous allograft plug into the opening of the interbody spacer, wherein the plug is configured to contact edges that define the opening when the cancellous allograft plug is inserted into the opening; and
inserting the vertebral spacer and cancellous allograft plug between two adjacent vertebrae to facilitate the fusion of the two adjacent vertebrae.

20. The method of claim 19, further comprising reconstituting the cancellous allograft plug in a material that enhances spinal fusion.

21. The method of claim 19, further comprising forming a plurality of ridges in the first and second opposing surfaces to prevent migration of the interbody spacer.

22. The method of claim 19, wherein the synthetic non-metallic radiolucent material is comprised of a thermoplastic material.

23. The method of claim 19, wherein the synthetic non-metallic radiolucent material is comprised of Polyetheretherketones (PEEK).

24. The method of claim 19, further comprising forming a plurality of radio opaque markers in the synthetic non-metallic radiolucent interbody spacer to allow a user to determine the position of the spinal fusion device relative to a spine using x-rays.

25. The method of claim 19, further comprising packaging the synthetic non-metallic radiolucent interbody spacer together with a corresponding cancellous allograft plug.
